(19) United States
(12) Patent Application Publication
(10) Pub. No.: US 2019/0275061 A1
(43) Pub. Date: Sep. 12, 2019

(54) INHIBITION OF FIBROSIS IN NON ALCOHOL FATTY LIVER DISEASE PATIENTS

(71) Applicant: GALMED RESEARCH AND DEVELOPMENT LTD, TEL AVIV (IL)

(72) Inventors: Liat Hayardeny-Nissimov, Tel Aviv (IL); Tali Gorfine, Tel Aviv (IL); Allen Baharaff, Tel Aviv (IL); Jose M. Mato De La Paz, Getxo (ES)

(73) Assignee: GALMED RESEARCH AND DEVELOPMENT LTD, Tel Aviv (IL)

(21) Appl. No.: 16/348,158

(22) PCT Filed: Nov. 10, 2017
(86) PCT No.: PCT/IB2017/001535

§ 371 Date: May 8, 2019

Related U.S. Application Data
(60) Provisional application No. 62/420,009, filed on Nov. 10, 2016.
(60) Provisional application No. 62/475,132, filed on Mar. 22, 2017.

(51) Int. Cl. A61K 31/575; A61K 9/00; A61P 1/16

(52) U.S. Cl. CPC A61K 31/575; A61P 1/16; A61K 9/0053

(57) ABSTRACT

The invention relates to inhibiting the development of hepatic fibrosis in a human subject afflicted with Non-Alcoholic Fatty Liver Disease and having a fibrosis score of zero comprising administering to the subject greater that 300 mg per day of 3β-arachidylamido-7α, 12α-dihydroxy-5β-cholan-24-oic acid (Aramchol), or a pharmaceutically acceptable salt thereof, thereby inhibiting the development of hepatic fibrosis in said subject.

This application claims the benefit of U.S. Provisional Application No. 62/475,132, filed Mar. 22, 2017 and U.S. Provisional Application Ho. 62/420,009, filed Nov. 10, 2016.

### BACKGROUND OF THE INVENTION

### Fibrosis

The formation of fibrous connective tissue is part of the normal healing process following tissue damage due to injury or inflammation. During this process, activated immune cells including macrophages stimulate the proliferation and activation of fibroblasts, which in turn deposit connective tissue. However, abnormal or excessive production of connective tissue may lead to accumulation of fibrous material such that it interferes with the normal function of the tissue. Fibrotic growth can proliferate and invade healthy surrounding tissue, even after the original injury heals. Such abnormal formation of excessive connective tissue, occurring in a reparative or reactive process, is referred to as fibrosis.

Fibrosis of the liver, also referred to herein as hepatic fibrosis, may be caused by various types of chronic liver injury, especially if an inflammatory component is involved. Self-limited, acute liver injury (e.g., acute viral hepatitis A), even when fulminant, does not necessarily distort the scaffolding architecture and hence does not typically cause fibrosis, despite loss of hepatocytes. However, factors such as chronic alcoholism, malnutrition, hemochromatosis, and exposure to poisons, toxins or drugs, may lead to chronic liver injury and hepatic fibrosis due to exposure to hepatotoxic chemical substances. Hepatic scarring, caused by surgery or other forms of injury associated with mechanical biliary obstruction, may also result in liver fibrosis.

Fibrosis itself is not necessarily symptomatic, however it can lead to the development of portal hypertension, in which scarring distorts blood flow through the liver, or cirrhosis, in which scarring results in disruption of normal hepatic architecture and liver dysfunction. The extant of each of these pathologies determines the clinical manifestation of hepato-fibrotic disorders. For example, congenital hepatic fibrosis affects portal vein branches, largely sparing the parenchyma. The result is portal hypertension with sparing of hepatocellular function.

### Treatment

In its initial stages, hepatic fibrosis may regress if the cause is reversible (e.g. with viral clearance). Thus, the majority of available treatment options are designed to remove the basis of the liver injury, such as by eliminating hepatitis B virus or hepatitis C virus in chronic viral hepatitis, abstaining from alcohol in alcoholic liver disease, removing heavy metals such as iron in hemochromatosis or copper in Wilson disease, and decompressing bile ducts in biliary obstruction.

Treatments aimed at reversing the fibrosis are usually too toxic for long-term use (e.g. corticosteroids, penicillamine) or have no proven efficacy (e.g. colchicine). Silymarin, present in milk thistle, is a popular alternative medicine used to treat hepatic fibrosis, appears to be safe but to lack efficacy. Potential Therapeutic agents

Attempts to develop specific anti-fibrotic agents for the treatment of liver diseases have been reported. For example, U.S. Pat. No. 8,729,046 relates to methods for treating fibrosis of a tissue, including fibrosis of the liver, using combinations of nucleic acids or nucleic acid analogs. Specifically, the nucleic acids or analogs thereof are targeted to microRNAs of the miR23b cluster. U.S. Pat. No. 6,562,829 discloses compositions for treating hepatic fibrosis and methods of using and manufacturing the composition, the composition comprising a quinazolinone derivative, preferably Halofuginone. U.S. Pat. No. 8,858,954 is directed to pharmaceutical composition for preventing and treating liver fibrosis or nonalcoholic fatty liver disease, comprising 50 to 90% by weight of Cordyceps sinensis mycelium powder, and 10 to 50% by weight of condensed astragalus powder.

U.S. Pub. No. 2015/359805 relates to Farnesoid X receptor (FXR) modulators which can be used for the treatment of cholestatic disorders. In particular to bile acid derivatives wherein the C6 contains an ethyl and the C24 carboxy group is transformed into a sulphate group. Among the disorders suggested to be treated are alcoholic liver disease, living donor transplant liver regeneration, congenital hepatic fibrosis, choledocholithiasis, and granulomatous liver disease. U.S. 2014/187633 is directed to methods of treating and/or preventing non-alcoholic seatohepatitis (NASH) and/or primary biliary cirrhosis comprising administering to a subject in need thereof a pharmaceutical composition comprising eicosapentaenoic acid or a derivative thereof. The FXR agonist, obeticholic acid, which is a modified bile acid, is in phase III clinical trials for primary biliary cirrhosis. Use of this drug has been reported to be commonly associated with side effects such as pruritus.

Ursodeoxycholic acid (UDCA, Ursodiol) is the most frequently used treatment for primary biliary cirrhosis. It is one of the secondary bile acids, which are metabolic byproducts of intestinal bacteria. The drug is considered to assist in reducing the cholestasis and improves blood test results (liver function tests). However it has a minimal effect on symptoms and whether it improves prognosis is controversial. To relieve itching caused by bile acids in circulation, which would normally be removed by the liver, cholestyramine (a bile acid sequestrant) may be prescribed to primary biliary cirrhosis patients. The agent may assist in absorbing bile acids in the gut to be eliminated, rather than re-enter the blood stream. Alternative agents include stanozolol, naltrexone and rifampicin.

Obeticholic acid (OCA, Ocaliva) is a semi-synthetic bile acid analogue undergoing development in phase 2 and 3 studies for specific liver and gastrointestinal conditions. The FDA granted accelerated approval to Ocaliva on 27 May 2016 for the treatment of primacy biliary cholangitis (PBC) in combination with ursodeoxycholic acid (UDCA) in adults with an inadequate response to UDCA, or aa a single therapy in adults unable to tolerate UDCA. In addition, a phase 2 trial in NASH patients shewed that administration of OCA reduced markers of liver inflammation and fibrosis and increased insulin sensitivity.

WO 2014/197736 and WO 2016/094570 relate to small molecule compounds, disclosed to be inhibitors of myofibroblast trans-differentiation and activation. Drugs and combinations suggested for the treatment of inter alia fatty liver were disclosed, for example, in WO 2016/112305 and EP2632925 (acetyl-CoA carboxylase inhibitors) as well as WO 2016/154258 (dual PPAR delta/gamma agonists). Some of the disclosed compounds wore suggested to be used in combination with various ether drugs.

Many patients do not respond to available treatments for fibrotic disorders, and long term treatment is limited by toxicity and side effects. Therefore, a need remains for developing therapeutic modalities aimed at reducing fibrosis, especially hepatic fibrosis. The development of safe and effective treatments for established cirrhosis and portal hypertension and for attenuating fibrosis would be highly beneficial.

### SUMMARY OF THE INVENTION

### Fatty Acid Bile Acid Conjugates

Fatty acid bile salt conjugates, referred to also as Fatty Acid Bile Acid Conjugated (FABACS), are a family oi synthetic molecules that may be used to improve conditions related to bile acids or cholesterol metabolism. FABACs are believed to lower blood cholesterol concentration, reduce liver fat levels and dissolve gallstones (Gilat et al., Hepatology 2003; 38: 436-442; and Gilat et al., Hepatology 2002; 35: 597-600). FABAC Include 3β-arachidylamido-7α, 12α-dihydroxy-5β-cholan-24-oic acid, also known as Aramchol.

U.S. Pat. Nos. 6,384,024, 6,395,722, 6,589,946 disclose certain FABACs and their use in dissolving cholesterol gallstones in bile and treating arteriosclerosis. These and additional FABACs were disclosed in U.S. Pat. Nos. 7,501,403, 8,975,246 and 8,110,564 for use in treating fatty liver, in reducing blood cholesterol levels and In treating hyperglycemia, diabetes, insulin resistance and obesity. Further therapeutic uses of FABACs are disclosed in Safadi et al. (Clin Gastroenterol Hepatol. 2014 December;12(12):2065-91) and in WO 2015/019358 and WO 2015/019359. Amine salts of certain FABACs are disclosed in wo 2015/093164.

The invention relates to inhibiting the development of hepatic fibrosis in a human subject afflicted with Non-Alcoholic Fatty Liver Disease and having a fibrosis score of zero comprising administering to the subject greater than 300 mg per day of 3β-arachidylamido-7α, 12α-dihydroxy-5β-cholan-24-oic acid (Aramchol), or a pharmaceutically acceptable salt thereof, thereby inhibiting the development of hepatic fibrosis in said subject.

The invention provides Aramchol and medicaments comprising Aramchol for use in any of the methods of the invention.

The invention relates to use of a therapeutically effective amount of 3β-arachidylamido-7α, 12α-dihydroxy-5β-cholan-24-oic acid (Aramchol), or a pharmaceutically acceptable salt thereof, for inhibiting the development of hepatic fibrosis in a human subject afflicted with Non-Alcoholic Fatty Liver Disease and having a fibrosis score of zero comprising administering to the subject greater than 300 mg per day of 3β-arachidylamido-7α, 12α-dihydroxy-5β-cholan-24-oic acid (Aramchol), or a pharmaceutically acceptable salt thereof, thereby inhibiting the development of hepatic fibrosis in said subject.

The invention provides 3β-arachidylamido-7α, 12α-dihydroxy-5β-cholan-24-oic acid (Aramchol), or a pharmaceutically acceptable salt thereof, for inhibiting the development of hepatic fibrosis in a human subject afflicted with Non-Alcoholic Fatty Liver Disease and having a fibrosis score of zero comprising administering to the subject greater than 300 mg per day of 3β-arachidylamido-7α, 12α-dihydroxy-5β-cholan-24-oic acid (Aramchol), or a pharmaceutically acceptable salt thereof, thereby for inhibiting the development of hepatic fibrosis in a human subject afflicted with Non-Alcoholic Patty Liver Disease and having a fibrosis score of zero comprising administering to the subject greater than 300 mg per day of 3β-arachidylamido-7α, 12α-dihydroxy-5β-cholan-24-oic acid (Aramchol), or a pharmaceutically acceptable salt thereof, thereby inhibiting the development of hepatic fibrosis in said subject.

The invention provides a medicament comprising 3β-arachidylamido-7α, 12α-dihydroxy-5β-cholan-24-oic acid (Aramchol), or a pharmaceutically acceptable salt thereof, for inhibiting the development of hepatic fibrosis in a human subject afflicted with Non-Alcoholic Fatty Liver Disease and having a fibrosis score of zero comprising administering to the subject greater than 300 mg per day of 3β-arachidylamido-7α, 12α-dihydroxy-5β-cholan-24-oic acid (Aramchol), or a pharmaceutically acceptable salt thereof, thereby for inhibiting the development of hepatic fibrosis in a human subject afflicted with Non-alcoholic Fatty Liver Disease and having a fibrosis score of zero comprising administering to the subject greater than 300 mg per day of 3β-arachidylamido-7α, 12α-dihydroxy-5β-cholan-24-oic acid (Aramchol), or a pharmaceutically acceptable salt thereof, thereby inhibiting the development of hepatic fibrosis In said subject.

The invention provides a pharmaceutical, package comprising a) 3β-arachidylamido-7α, 12α-dihydroxy-5β-cholan-24-oic acid (Aramchol), or a pharmaceutically acceptable salt thereof; and b) instructions for use in inhibiting the development of hepatic fibrosis in a human subject afflicted with Non-Alcoholic Fatty Liver Disease and having a fibrosis score of zero comprising administering to the subject greater than 300 mg per day of 3β-arachidylamido-7α, 12α-dihydroxy-5β-cholan-24-oic acid (Aramchol), or a pharmaceutically acceptable salt thereof, thereby inhibiting the development of hepatic fibrosis in said sublet.

Other objects, features and advantages of the present invention will become clear from the following description and drawings.

### BRIEF DESCRIPTION OF THE DRAWINGS

FIG. 1. demonstrates the effect of Aramchol on liver cirrhosis by macroscopic evaluation. FIG. 1A—saline control; FIG. 1B—treatment with TAA (20 mg/100 gr body weight) twice per week during 10 weeks; FIG. 1C—treatment with TAA and Aramchol 1 mg/kg; FIG. 1D—treatment with TAA and Aramchol 5 mg/kg.

FIG. 2. demonstrates the effect of Aramchol on liver fibrosis by microscopic evaluation (following Kasson Goldner staining). FIG. 2A—averaged fibrotic score (TAA—black, Aramchol—white, OCA—gray); FIG. 2B—representative samples (TAA only—left; TAA and Aramchol 1 mg/kg—middle; TAA and Aramchol 5 mg/kg—right).

FIG. 3. depicts the effect of Aramchol on COL1A1 expression in LX-2 human hepatic stellate cells. “Ctrl S1, S2 and S3” represent control (saline-treated cells) in three separate experiments; “A S1, S2 and S3” represent the result of Aramchol treated cells in these experiments.

FIG. 4. depicts the effect of Aramchol on PPAR-γ expression in LX-2 human hepatic stellate cells. “Ctrl S1, S2 and S3” represent control (saline-treated cells) in three separate experiments; “A S1, S2 and S3” represent the result of Aramchol treated cells in these experiments.

FIG. 5. depicts the effect of Aramchol on collagen production from LX-2 human hepatic stellate cells compared to a DMSO control.

FIG. 6. depicts the effect of Aramchol on liver steatosis in 0.1 MCD diet. FIG. 6A—histology staining using Sudan III; FIG. 6B—quantification of Sudan III stained cells.

FIG. 7. depicts the effect of Aramchol on macrophage activation and infiltration in 0.1 MCD diet. FIG. 7A—histology staining—F4/80 and CD64; FIG. 7B—quantification of F4/80 and CD64 positive cells.

FIG. 8. depicts the effect of Aramchol on fibrosis in 0.1 MCD Diet (histology—sirius red). FIG. 8A—histology staining using sirius red; FIG. 8B—quantification of sirius red stained cells.

FIG. 9. depicts the effect of Aramchol on collagen production using liver extract free 0.1 MCD size.

FIG. 10. depicts the effects of Aramchol on liver biochemistry in 0.1 MCD mice. FIG. 10A—quantification of metabolites in liver of control (grey) and Aramchol-treated (black) 0.1 MCD mice; FIG. 10B—schematic of relevant liver biochemical pathway.

### DETAILED DESCRIPTION OF THE INVENTION

### Aramchol

Aramchol is chemically named 3β-arachidylamido-7α, 12α-dihydroxy-5β-cholan-24-oic acid, and is represented by the following chemical structure:

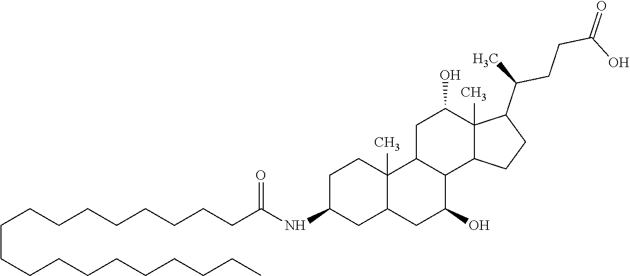 [SMILES: CCCCCCCCCCCCCCCCCCCC(=O)N[C@H]1CCC2(C)C(C1)C[C@H](O)C1C2C[C@H](O)C2(C)C1CCC2[C@@H](C)CCC(=O)O]

According to an embodiment off the invention, the combinations, compositions, methods and packages of the invention may comprise Aramchol in its free acid form. According to an embodiment of the invention, Aramchol is in its salt form. The salt may be an amine-based salt. The amine-based salt may be selected from the group consisting of meglumine, lysine and tromethamine salts.

Other embodiments of the invention relate to compositions, methods and packages employing the use of a Fatty Acid Bile Acid Conjugate (FABAC), or salts thereof. According to some embodiments, the FABAC is of Formula 1:

W-X-G (I)

wherein G represents a bile acid or a bile salt radical thereof; W represents one or two fatty acid radicals having 6-22 carbon atoms; and X represents a bonding member selected from the group consisting of: a heteroatom, a direct C—C bond and a C═C bond. Each possibility represents a separate embodiment of the present invention.

According to some embodiments, the bonding member is selected from the group consisting of: NH, P, S, O and a direct C—C or C═C bond. Each possibility represents a separate embodiment, of the present invention. According to some embodiments, the bonding member is NH.

According to some embodiments, each of said one or two fatty acid radicals is a radical of a fatty acid selected from the group consisting of: arachidylic acid, stearic acid, behenic acid, palmitic acid, arachidonic acid, eicosapentaenoic acid and oleic acid. Each possibility represents a separate embodiment of the present invention. According to some embodiments, said one or two fatty acid radicals are radicals of arachidylic acid. Each possibility represents a separate embodiment of the present invention.

According to some embodiments, W represents two fatty acid radicals, each independently comprises 6-22 carbon atoms; and wherein each of said fatty acid radicals is independently bound to a bonding member X selected from the group consisting of: a heteroatom, a direct C—C bond and a C═C bond. According to some embodiments, W represents a single fatty acid radical.

According to some embodiments, the bile acid is selected from the group consisting of: cholic acid, ursodeoxycholic acid, chenodeoxycholic acid, deoxycholic acid, lithocholic acid and derivatives thereof. Each possibility represents a separate embodiment of the present invention. In another embodiment the bile acid is cholic acid, chenodeoxycholic acid, or deoxycholic acid. In another embodiment the bile acid is other than ursodeoxycholic acid and lithocholic acid. According to some embodiments, the bile acid is cholic acid.

### Indications

The invention is based, in part, on the surprising discovery that Aramchol exerts a potent anti-fibrotic effect, independent of its reported activities on fatty liver and steatosis, and inhibits the development of fibrosis in various experimental models. Specifically, treatment with Aramchol (5 mg/kg) significantly inhibited the development of toxin-induced cirrhosis, necrosis and liver fibrosis in an in vivo thioecetamide (TAA) model. Aramchol was also found to be unexpectedly superior to obaticholic acid (OCA), which did not induce statistically significant reduction in these parameters under the tested experimental conditions. In addition, Aramchol significantly reduced COL1A1 expression in LX-2 human hepatic stellate cello via PPARγ up-regulation.

Thus, independently from its reported activities on liver metabolism in subjects with NAFLD, Aramchol is surprisingly found herein to be effective in the treatment of new patient populations and patient sub-populations, such as in the treatment of patients with NAFLD or NASH who have not yet developed fibrosis. The invention advantageously provides for the treatment of these now patient populations with enhanced efficacy and/or safety and minimized side effects.

This invention provides a not bod for inhibiting the development of hepatic fibrosis in a human subject afflicted with Non-Alcoholic Fatty Liver Disease and having a fibrosis score of zero comprising administering to the subject greater than 300 mg per day of 3β-arachidylamido-7α, 12α-dihydroxy-5β-cholan-24-oic acid (Aramchol), or a pharmaceutical acceptable salt thereof, thereby inhibiting the development of hepatic fibrosis in said subject. In an embodiment the human subject being treated la afflicted with Non-Alcoholic Fatty Liver Disease (NAFLD).

In an embodiment the human subject being treated is afflicted with Non-Alcoholic Steatohepatitis (NASH).

In an embodiment the human subject is afflicted with NAFLD but not afflicted with Non-Alcoholic Steatohepatitis (NASH). In an embodiment the human subject has a NAFLD Activity (NAS) Score of at least 4. In an embodiment the human subject has a NAFLD Activity (NAS) Score of at least 5, at least 6, or at least 7. In an embodiment the human subject has a ballooning score of at leant 1, an inflammation score of at least 1, and a steatosis score of at least 1.

In an embodiment the human subject is afflicted with NAFLD but not afflicted with Non-Alcoholic Steatohepatitis (NASH).

In an embodiment the human subject is afflicted with Diabetes Mellitus type II or pre-diabetes. One of the following 3 criteria is needed for pre-Diabetes: Fasting Plasma Glucose>100 mg/dl (5.5 mmol/l) or 2hPG following 75 g OGTT>140 (7.8 mmol/l) mg/dl or HbA1c>5.7%. HbA1c can be repeated at Investigator's discretion.

In an embodiment the human subject has a diet that is high fat and high calorie. As used herein, a high fat, high calorie diet contains at least 4000 calories per day, of which approximately 50% comes from fat.

In an embodiment the human subject is resistant to lifestyle intervention.

In an embodiment the human subject is resistant to diet intervention.

### Administration and Dosage Form

According to some embodiments, the compound to be administered (e.g. Aramchol) is in the form of a composition (referred to as the composition of the invention) comprising a therapeutically effective amount of at least one of said compound. As used herein, the terra “effective amount” means an amount of compound that is capable of reducing and/or attenuating a disorder or symptom as described herein. The specific dose of a compound administered according to this invention will, of course, be determined by the particular circumstances surrounding the case including, for example, the compound administered, the route of administration, the physiological state of the subject, and the severity of the condition being treated.

This invention provides a medicament comprising greater than 300 mg of 3β-arachidylamido-7α, 12α-dihydroxy-5β-cholan-24-oic acid (Aramchol) for use in administration to a human subject, including any of the human subjects recited hereinabove.

In an embodiment the medicament comprises greater than 350 mg of Aramchol. In an embodiment the medicament comprises between 350 mg and 1200 mg of Aramchol. In an embodiment the medicament comprises 400 mg, 500 mg, 600 mg, 700 mg, 800mg, 900 mg, 1000 mg, 1100 mg, or 1200 mg of Aramchol. In an embodiment the medicament comprises between 400 mg and 1100 mg, or between 500 mg and 1000 mg, or 600 mg and 900 mg of Aramchol. In an embodiment the medicament comprises 400 mg or 600 mg of Aramchol.

In an embodiment the medicament is to be administered daily.

This invention also provides 3β-arachidylamido-7α, 12α-dihydroxy-5β-cholan-24-oic acid (Aramchol) for use in administration to a human subject at a daily dose of greater than 300 mg. In an embodiment the human subject is any of the human subjects recited hereinabove.

In an embodiment the daily dose of Aramchol is greater than 350 mg. In an embodiment the daily dose of Aramchol is between 350 mg and 1200 mg. In an embodiment the daily dose of Aramchol is 400 mg, 500 mg, 600 mg, 700 mg, 800 mg, 900 mg, 1000 mg, 1100 mg, or 1200 mg. In an embodiment the daily dose of Aramchol is between 400 mg and 1100 mg, or between 500 mg and 1000 mg, or between 600 mg and 900 mg. In an embodiment the daily dose of Aramchol is 400 mg or 600 mg per day.

Any suitable route may be used to administer the medicament or Aramchol of the invention to a subject.

According to some embodiments, suitable administration routes may be systemic routes. According to some embodiments, administering is administering systemically. According to some embodiments, the composition is formulated for systemic administration.

In another embodiment of the methods of the invention, Aramchol is in the form of Aramchol free acid. In another embodiment of the methods of the invention, Aramchol is in the form of an amine-based salt. In certain particular embodiments, the salt is a meglumine, lysine or tromethamine Aramchol salt. Each possibility represents a separate embodiment of the invention.

According to another embodiment, administration systemically is through an enteral route. According to another embodiment, administration through an enteral route is oral administration. According to some embodiments, the composition is formulated for oral administration.

Thus, the inventions provides a method for treating the subjects recited in this application by administering Aramchol to the subject, wherein at least 350 mg of Aramchol is administered to the subject per day. In an embodiment between 350 mg and 1200 mg of Aramchol is administered to the subject per day. In an embodiment 400 mg, 500 mg, 600 mg, 700 mg, 800 mg, 900 mg, 1000 mg, 1100 mg, or 1200 mg of Aramchol is administered to the subject per day. In an embodiment between 400 mg and 1100 mg, or between 500 mg and 1000 mg, or between 600 mg and 900 mg of Aramchol is administered to the subject per day. In an embodiment 400 mg or 600 mg of Aramchol is administered to the subject per day.

In an embodiment the medicament of Aramchol is administered in the morning, in the afternoon, or in the evening.

In an embodiment the medicament or Aramchol is administered at the same time as, or within 30 minutes of a meal.

In an embodiment the meal is breakfast, lunch, or dinner.

In an embodiment the meal is a high fat meal. A high fat meal is a meal wherein approximately 500 to 600 calories are fat calories.

In an embodiment the meal its a high calorie meal. A high calorie meal is a meal of approximately 800 to 1000 calories.

In an embodiment the medicament or Aramchol is administered with water. In an embodiment the medicament or Aramchol is administered with at least 100 or at least 200 mL of water.

In an embodiment the Aramchol is administered over the course of at least 32 weeks, at least 72 weeks, at least 96 weeks, at least 2 years, at least 3 years, or at least 4 years.

The invention relates to use of a therapeutically effective amount of 3β-arachidylamido-7α, 12α-dihydroxy-5β-cholan-24-oic acid (Aramchol), or a pharmaceutically acceptable salt thereof, for inhibiting the development of hepatic fibrosis in a human subject afflicted with Non-Alcoholic Fatty Liver Disease and having a fibrosis score of zero comprising administering to the subject greater than 300 mg per day of 3β-arachidylamido-7α, 12α-dihydroxy-5β-cholan-24-oic acid (Aramchol), or a pharmaceutically acceptable salt thereof, thereby inhibiting the development of hepatic fibrosis in said subject.

The invention provides 3β-arachidylamido-7α, 12α-dihydroxy-5β-cholan-24-oic acid (Aramchol), or a pharmaceutically acceptable salt thereof, for inhibiting the development of hepatic fibrosis in a human subject afflicted with Non-Alcoholic Fatty Liver Disease and having a fibrosis score of zero comprising administering to the subject greater than 300 mg per day of 3β-arachidylamido-7α, 12α-dihydroxy-5β-cholan-24-oic acid (Aramchol), or a pharmaceutically acceptable salt thereof, thereby for inhibiting the development of hepatic fibrosis in a human subject afflicted with Non-Alcoholic Fatty Liver Disease and having a fibrosis score of zero comprising administering to the subject greater than 300 mg per day of 3β-arachidylamido-7α, 12α-dihydroxy-5β-cholan-24-oic acid (Aramchol), or a pharmaceutically acceptable salt thereof, thereby inhibiting the development of hepatic fibrosis in said subject.

The invention provides a medicament comprising 3β-arachidylamido-7α, 12α-dihydroxy-5β-cholan-24-oic acid (Aramchol), or a pharmaceutically acceptable salt thereof, tor inhibiting the development of hepatic fibrosis in a human subject afflicted with Non-Alcoholic Fatty Liver Disease and having a fibrosis score of zero comprising administering to the subject greater than 300 mg per day of 3β-arachidylamido-7α, 12α-dihydroxy-5β-cholan-24-oic acid (Aramchol), or a pharmaceutically acceptable salt thereof, thereby for inhibiting the development of hepatic fibrosis in a human subject afflicted with Non-Alcoholic Fatty Liver Disease and having a fibrosis score of zero comprising administering to the subject greater than 300 mg per day of 3β-arachidylamido-7α, 12α-dihydroxy-5β-cholan-24-oic acid (Aramchol), or a pharmaceutically acceptable salt thereof, thereby Inhibiting the development of hepatic fibrosis in said subject.

The invention provides a pharmaceutical package comprising a) 3β-arachidylamido-7α, 12α-dihydroxy-5β-cholan-24-oic acid (Aramchol), or a pharmaceutically acceptable salt thereof; and b) instructions for use in inhibiting the development of hepatic fibrosis in a human subject afflicted with Non-Alcoholic Fatty Liver Disease and having a fibrosis score of zero comprising administering to the subject greater than 300 mg per day of 3β-arachidylamido-7α, 12α-dihydroxy-5β-cholan-24-oic acid (Aramchol), or a pharmaceutically acceptable salt thereof, thereby inhibiting the development of hepatic fibrosis in said subject.

According to some embodiments, oral administration is in the form of hard or soft gelatin capsules, pills, capsules, tablets, including coated tablets, dragees, elixirs, suspensions, liquids, gels, slurries or syrups and controlled release forms thereof. Thus the invention provides a method of administering Aramchol in the form of a tablet, a capsule, or in a liquid.

Suitable carriers for oral administration are well known in the art. Compositions for oral use can be made using a solid excipient, optionally grinding the resulting mixture, and processing the mixture of granules, after adding suitable auxiliaries as desired, to obtain tablets or dragee cores. Non-limiting examples of suitable excipients include fillers such as sugars, including lactose, sucrose, mannitol, or sorbitol, cellulose preparations such as, maize starch, wheat starch, rice starch, potato starch, gelatin, gum tragacanth, methyl cellulose, hydroxypropylmethyl-cellulose, and sodium carbomethylcellulose, and/or physiologically acceptable polymers such as polyvinylpyrrolidone (PVP).

If desired, disintegrating agents, such as cross-linked polyvinyl pyrrolidone, agar, or alginic acid or a salt thereof, such as sodium alginate, may be added. Capsules and cartridges of, for example, gelatin for use in a dispenser may be formulated containing a powder mix of the compound and a suitable powder base, such as lactose or starch.

Solid dosage forms for oral administration include without limitation capsules, tablets, pills, powders, and granules. In such solid dosage forms, the active compound is admixed with at least one inert pharmaceutically acceptable carrier such as sucrose, lactose, or starch. Such dosage forms can also comprise, as it normal practice, additional substances other than inert diluents, e.g., lubricating, agents. In the case of capsules, tablets and pills, the dosage forms may also comprise buffering agents. Tablets and pills can additionally be prepared with enteric coatings. The term “enteric coating”, as used herein, refers to a coating which controls the location of composition absorption within the digestive system. Non-limiting examples for materials used for enteric coating are fatty acids, waxes, plant fibers or plastics.

Liquid dosage forms for oral administration may further contain adjuvants, such as wetting agents, emulsifying and suspending agents, and sweetening, flavoring and perfuming agents.

According to some embodiments, concomitant treatment with fatty acids such as ethyl eicosapentanoate, eicosapentaenoic acid, and their amides, salts and phospholipids is explicitly excluded. In other embodiment, concomitant treatment with bile acids such as ursodeoxycholic acid and lithocholic acid is excluded. In other embodiments concomitant treatment with vitamin D receptor agonists, acetyl-CoA carboxylase inhibitors, dual PPAR delta/gamma agonists, and inhibitors of myofibroblast trans-differentiation and activation is excluded. According to advantageous embodiments, Aramchol or the pharmaceutically acceptable salt thereof is used as a sole active ingredient.

According to some embodiments, the composition is administered in several dosages over prolonged periods until a sufficient response has been achieved.

As disclosed herein, Aramchol was found to be an unexpectedly potent therapeutic agent, capable of inhibiting the development of fibrosis and reducing likelihood of cirrhosis and collagen synthesis in stellate cells even when used as a single therapeutic agent, in the absence of adjunct therapy. Thus, according to an advantageous embodiment of the methods of the invention, Aramchol or the pharmaceutically acceptable salt thereof is administered as a sole active ingredient. In another embodiment, the subject is human.

### Patient Outcomes

In an embodiment the administration of Aramchol or medicament comprising Aramchol prevents worsening of the subject's NAFLD Activity (NAS) score.

In an embodiment the administration of Aramchol or medicament comprising Aramchol prevents worsening of the subject's Steatosis, Activity and Fibrosis (SAF) Activity score.

In an embodiment inhibiting the development of hepatic fibrosis comprises prevention of worsening of fibrosis score.

In an embodiment prevention is prevention at 52, 65, 72 or 96 weeks from the commencement of administration of Aramchol.

In an embodiment prevention is prevention at 2, 3, or 4 years from the commencement of administration of Aramchol.

In an embodiment the administration of Aramchol or medicament comprising Aramchol improves the subject's NAFLD Activity (NAS) score.

In an embodiment the subject's MAS score is at least 4 at the commencement of administration of Aramchol or medicament comprising Aramchol and the improvement of the subject's NAS score is an improvement of at least 2 points.

In an embodiment the administration of Aramchol or medicament comprising Aramchol improves the subject's Steatosis, Activity and Fibrosis (SAF) Activity Score.

In an embodiment the subject's SAF Activity score is at least 4 at the commencement of administration of Aramchol or medicament comprising Aramchol and improvement of the subject's SAP Activity score is an improvement of at least 2 points.

In an embodiment improvement is Improvement at 52, 65, 12, or 96 weeks from the commencement of administration of Aramchol.

In an embodiment improvement is improvement, at 2, 3, or 4 years from the commencement of administration of Aramchol.

In an embodiment inhibiting the development of hepatic fibrosis comprises reducing progression of hepatic fibrosis relative to a patient not treated with Aramchol.

In an embodiment the administration of Aramchol or medicament comprising Aramchol inhibits progression of Non-Alcoholic Fatty Liver Disease (NAFLD).

In an embodiment inhibiting progression of NAFLD comprises prevention of progression, or reduced progression relative to a patient not treated with Aramchol.

In an embodiment the human subject is afflicted with Non-Alcoholic Steatohepatitis (NASH) and that administration of Aramchol inhibits progression of NASH.

In an embodiment inhibiting progression of NASH comprises prevention of progression, or reduced progression relative to a patient not treated with Aramchol.

In an embodiment the human subject is not afflicted with Non-Alcoholic Steatohepatitis (NASH) at commencement of administration and the administration of Aramchol inhibits progression from Non-Alcoholic Fatty Liver Disease (NAFLD) to NASH.

In an embodiment progression is progression at 52, 65, 72, or 96 weeks from the commencement of administration of Aramchol.

In an embodiment progression is progression at 2, 3, or 4 years from the commencement of administration of Aramchol.

In an embodiment the human subject is afflicted with Non-Alcoholic Steatohepatitis (NASH) and the administration of Aramchol resolves NASH in the subject.

In an embodiment NASH resolution comprises the human subject having a ballooning score of 0 and an inflammation score of 0 or 1.

In an embodiment the administration of Aramchol or medicament comprising Aramchol resolves NASH in the subject at 52, 65, 12, or 96 weeks from the commencement of administration.

In an embodiment the administration of Aramchol or medicament comprising Aramchol resolves NASH in the subject at 2, 3, or 4 years from the commencement of administration.

In an embodiment the administration of Aramchol or medicament comprising Aramchol reduces the level of liver triglycerides in the subject relative to the level at the commencement of administration of Aramchol.

In an embodiment the administration of Aramchol or medicament comprising Aramchol reduces the ratio of livor triglycerides to water in the subject relative to the ratio at the commencement of administration of Aramchol.

In an embodiment there is a greater than 10% reduction in ratio of liver triglycerides to water.

In an embodiment there is a 10% to 40% reduction in ratio of liver triglycerides to water.

In an embodiment there is a 15% to 35% reduction in ratio of liver triglycerides to water.

In an embodiment there is a 20% to 30% reduction in ratio of liver triglycerides to water.

In an embodiment the administration of Aramchol or medicament comprising Aramchol:
a. reduces the level of Hemoglobin A1C or HOMA-IR;
b. reduces the level of Fibrinogen, CK-18, c-reactive protein (CRP), TNFα, IL 6 and fibrosis Tests (NFS;
c. reduces the ratio of leptin to adinopectin; or
d. increase in the level of adinopectin;
   in the subject relative to the level or ratio at the commencement of administration.

In an embodiment the administration of Aramchol or medicament comprising Aramchol:
a. reduces the human subject's body weight relative to the human subject's body weight ac the commencement of administration of Aramchol;
b. reduces the human subject's waist circumference relative to the human subject's waist circumference at the commencement of administration of Aramchol; or
c. reduces the human subject's Fatty Liver Index relative to the human subject's Fatty Liver Index at the commencement of administration of Aramchol.

In an embodiment the reduction or increase is a reduction or increase at 2, 4, 8, 24, 40, 52, 65, 72, or 96 weeks from the commencement of administration.

In an embodiment the reduction or increase is a reduction or increase at 2, 3, or 4 years free the commencement of administration.

Thus, in various embodiments medicaments or Aramchol are contemplated that are effective to achieve the outcomes described above. In an embodiment the medicament or Aramchol is effective at 2, 4, 8, 24, 40, 52, 65, 72, or 96 weeks, or 2 years, 3 years, or 4 years from the commencement of administration.

In another embodiment, administration of Aramchol or a pharmaceutically acceptable salt thereof according to the methods of the invention inhibits collagen synthesis (e.g. COL1A1 expression) in hepatic stellate calls. In another embodiment, administration of Aramchol or a pharmaceutically acceptable salt thereof according to the methods of the invention enhances PPAR-γ expression in hepatic stellate calls.

It is understood that where a parameter range is provided, all integers within that range, and tenths thereof, are also provided by the invention. For example, “0.2-5 mg/kg/day” is a disclosure of 0.2 mg/kg/day, 0.3 mg/kg/day, 0.4 mg/kg/day, 0.5 mg/kg/day, 0.6 mg/kg/day etc. up to 5.0 mg/kg/day.

Each embodiment disclosed herein is contemplated as being applicable to each of the other disclosed embodiments. Thus, all combinations of the various elements described herein are within the scope of the invention.

The following examples are presented in order to more fully illustrate some embodiments of the invention. They should, in no way be construed, however, as limiting the broad scope of the invention.

### EXAMPLES

Examples are provided below to facilitate a more complete understanding of the invention. The following examples illustrate the exemplary nodes of making and practicing the invention. However, the scope of the invention is not limited to specific embodiments disclosed in these Examples, which are for purposes of illustration only.

### Example 1

### Thioacetamide (TAA)-Induced Fibrosis—Model for Hepatic Cirrhosis

Liver fibrosis was induces in Wistar rats by intraperitoneal injections of TAA 120 mg/100 gr body weight) twice per week during 10 weeks. I.p. application of TAA results in hepatic centrolobular necrosis, elevated transaminase activity and robust liver fibrosis. Treatment groups further included co-administration of Aramchol (1 or 5 mg/kg orally) or obaticholic acid (OCA, 5 mg/kg). A control group of saline-treated rats (in the absence of TAA administration) was further included. Rats were then sacrificed, and livers were observed macroscopically for signs of cirrhosis and necrotic lesions, and microscopically, following Masson Goldner staining. The fibrosis score, calculated at a scale of 0-4, was determined for each sample, wherein 0 indicates no fibrosis and 4 indicates advanced fibrosis and cirrhosis.

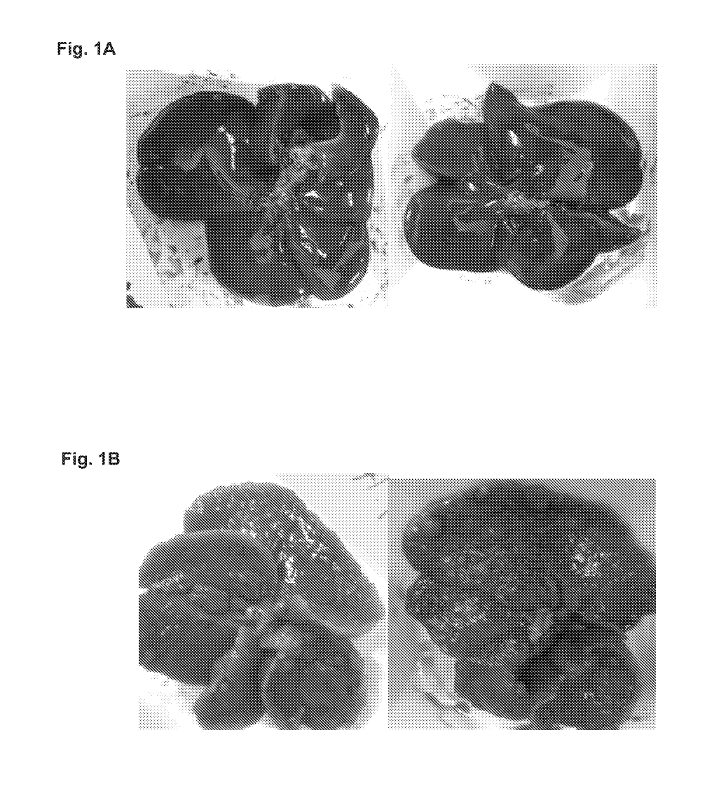

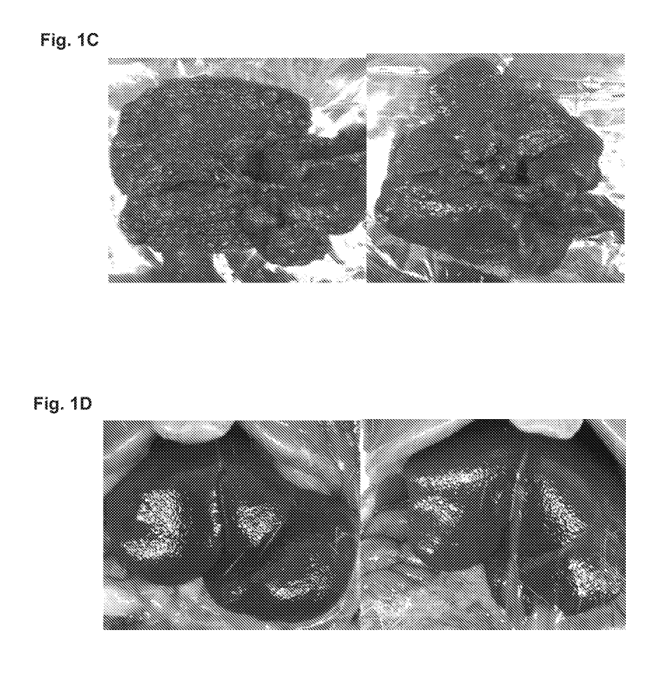

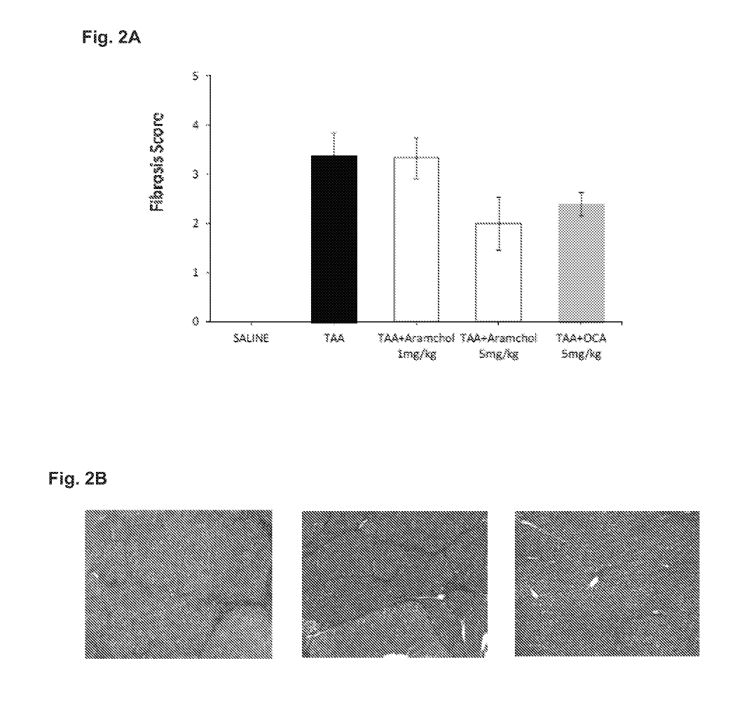

As can be seen in FIGS. 1-2, treatment with Aramchol (5 mg/kg) significantly prevented TAA induced fibrosis. The treatment reduced significantly the development of necrosis and cirrhosis (FIG. 1), as well as the fibrotic score and collagen distribution in the tissue (FIG. 2), in a dose-dependent manner. In contradistinction, OCA did not induce statistically significant reduction in these parameters.

Thus, Aramchol was surprisingly found to be a potent anti-fibrotic and anti-cirrhotic agent. Aramchol was also found to be unexpectedly superior to OCA and provide for improved, effective treatment for liver fibrosis.

Cirrhosis and portal hypertension from TAA intoxication may eventually lead to the development, of acute liver failure and associated conditions such as hepatic encephalopathy, and the TAA model is also used in evaluating these phenomena. Accordingly, as disclosed herein, Aramchol may also be used in some embodiments for preventing acute or fatal liver failure and/or hepatic or portosystemic encephalopathy, for example toxin-induced liver failure and/or hepatic encephalopathy.

### Example 2

### Inhibition of Collagen Synthesis in Stellate Cells

LX2 cells (150.000 cells per well) were plated in DMEM media containing antibiotics, glutamine and bovine fetal serum. After 24 hours incubation, media was changed to 0% serum and incubated for an additional period of 16 hours. Then, Aramchol (10 mM) was added and 24 hours later RNA was extracted with Trizol.

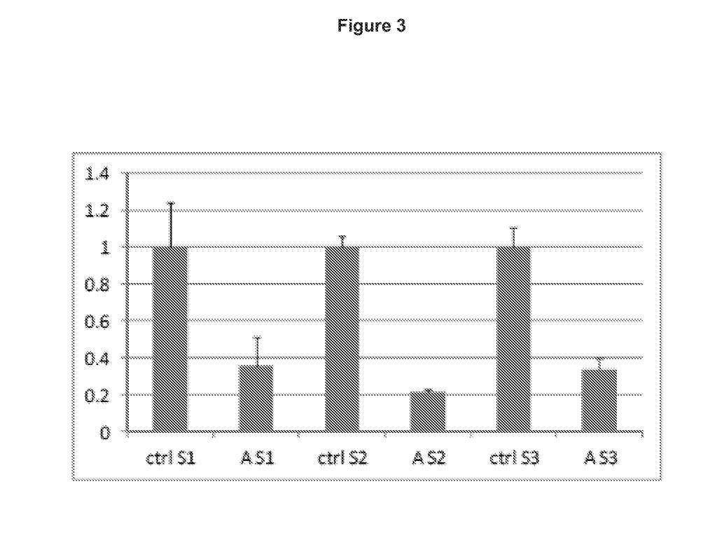

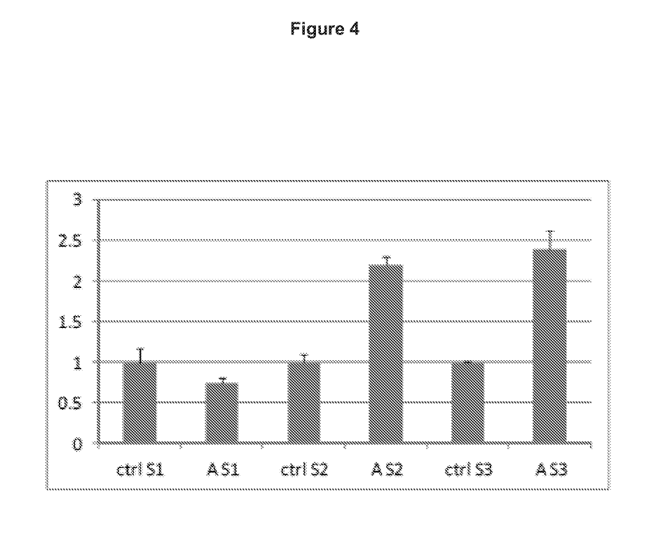

Surprisingly, as can be seen in FIGS. 3 and 4, COL1A1 expression in LX-2 human hepatic stellate cells was reduced by Aramchol via PPARγ up-regulation.

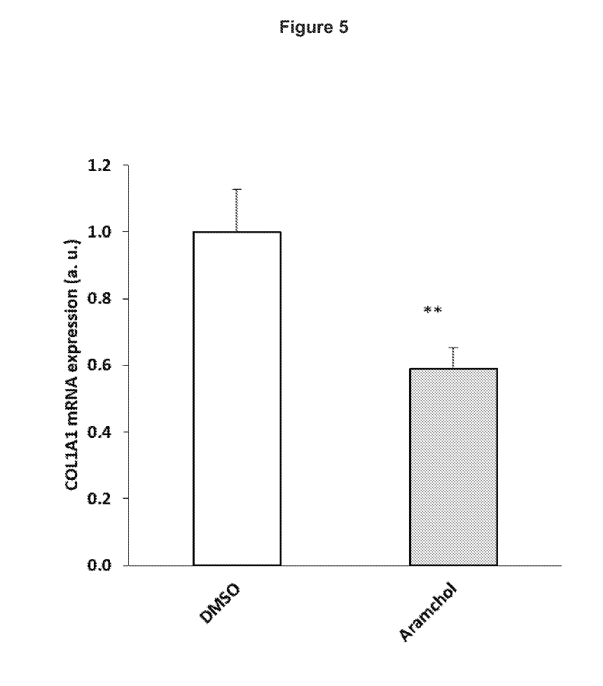

Consistently, Aramchol significantly down regulates collagen production in LX-2 human hepatic stellate cells relative to a DMSO control (FIG. 5). Again, Aramchol was surprisingly found to be effective in reducing the production of collagen specifically in stellate cells.

### Example 3

### Aramchol Reduces Established Fibrosis in a MCD Diet Animal Model

The study described below investigates the mechanism of action of Aramchol and its potential effect on fibrosis using the 0.1% methionine- and choline-deficient (0.1 MCD) diet mouse model of NASH.

C57B1/6 were fed the Methionine and Choline Deficient (MCD) and control diet and were sacrificed after 4 weeks. The MCD diet induces aminotransferase elevation and changes in hepatic histological features, characterized by steatosis, local inflammation, hepatocyte necrosis and fibrosis. These changes occur rapidly and are morphologically similar to those observed in human NASH. In this study the MCD diet contained 0.1% methionine to minimize and stabilize weight loss. At the end of the second week, after verification of established NASH, 0.1 MCD-fed mice were treated orally by gavage with Aramchol (5 mg/Kg/day) or vehicle (n=10, each condition). Control diet-fed mice were also treated with vehicle for same duration (n=10). At the end of the experiment, blood and liver samples were obtained. A diagram of the experimental design is shown below:

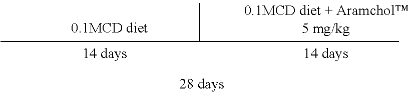

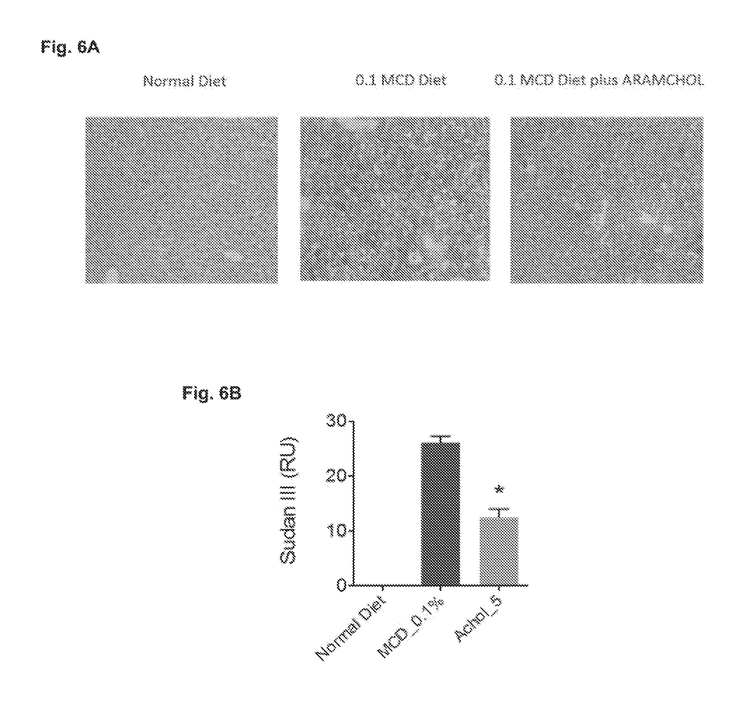

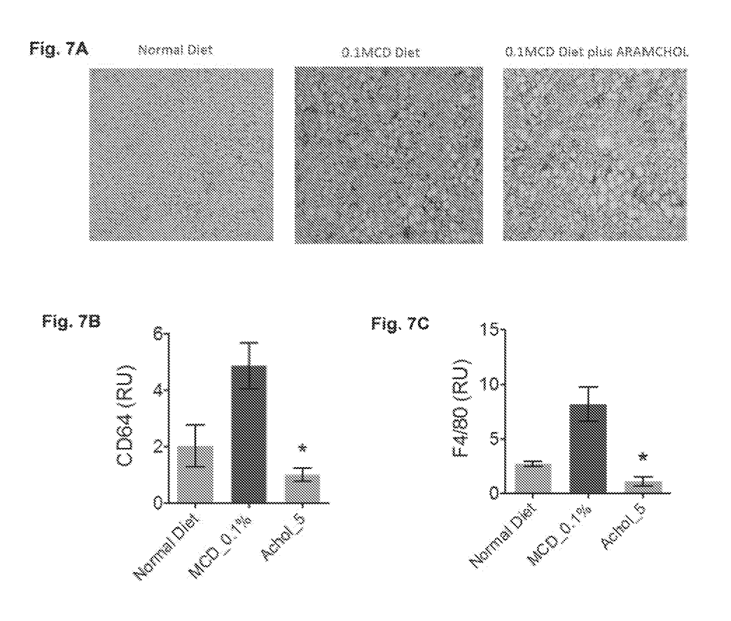

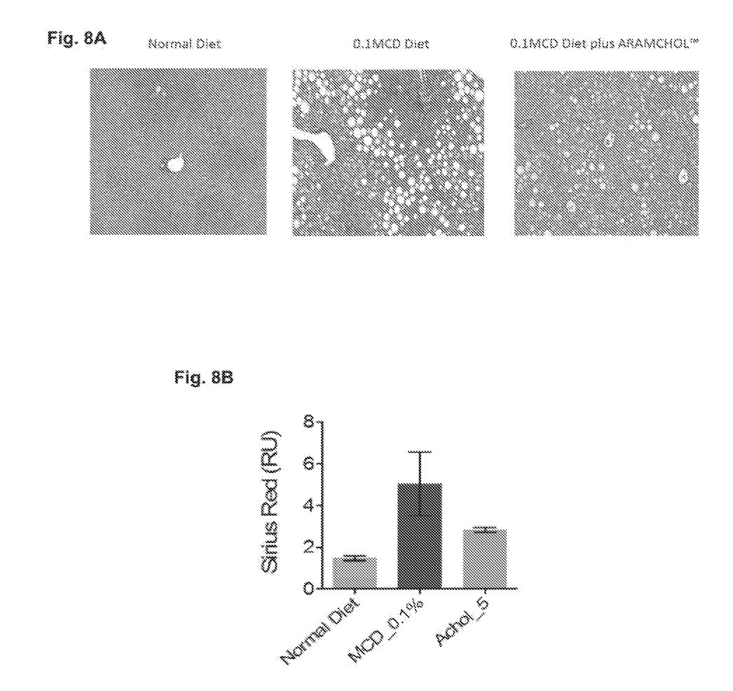

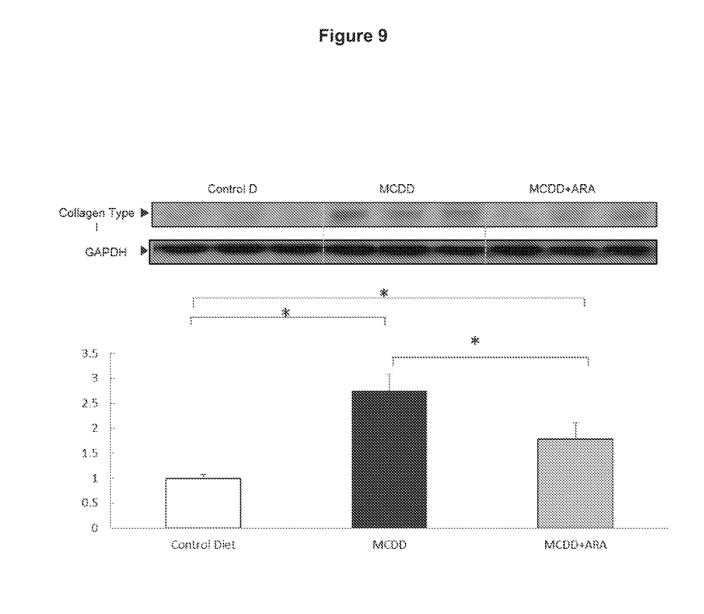

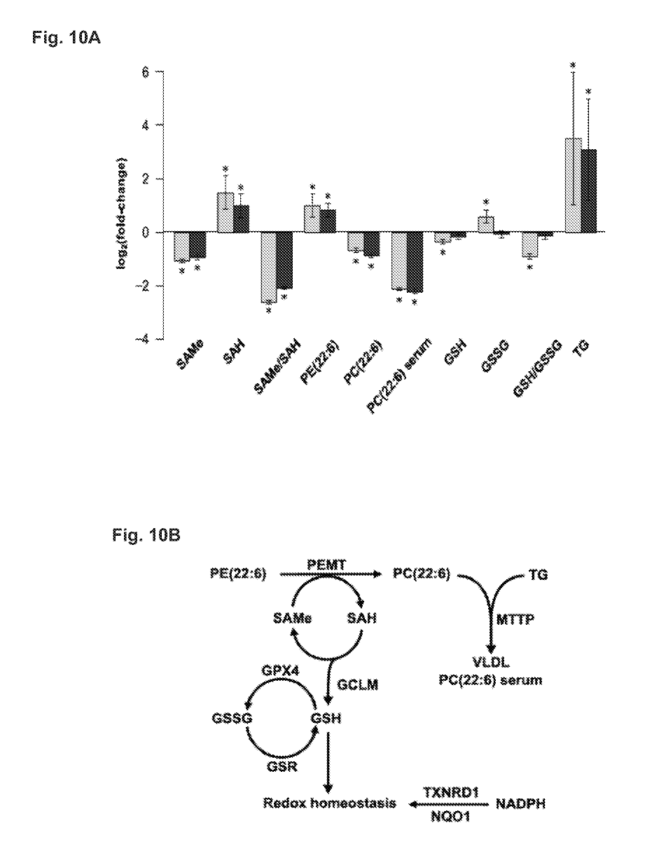

Results from the study showed: 1) treatment with Aramchol significantly down regulates steatosis in the liver (FIG. 6); 2) treatment with Aramchol significantly down regulates/normalizes infiltration and activation status of macrophages in the liver (FIG. 7); 3) treatment with Aramchol significantly down regulates/normalizes fibrosis in the liver (FIG. 8); 4) Aramchol significantly down regulates collagen in the liver (FIG. 9); and 5) Aramchol significantly up regulates glutathione and elevates GSH/GSSG ratio in 0.1 % MCD mice (FIG. 10).

Additionally, Aramchol treatment further reduced SCD1 activity, which was evidenced by a marked decrease in SCD1 expression, in the FA(16:1)/FA(16:0) ratio and in the total content of monounsaturated FA (MUFA), which led to a reduction in the hepatic content of diglycerides (DG) and TG. Aramchol treatment improved oxidative stress, as shown by the normalization of the GSH/GSSG ratio, a biomarker of the cellular redox potential, and a marked reduction in the content of total oxFA including oxLA, which has been associated with liver injury in human NASH.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to he comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

### Example 4

### Brief Summary

This is a multicenter, Phase IIb, randomized, double blind, placebo-controlled study designed to evaluate the efficacy and safety of two Aramchol doses in subjects that are 18 to 75 years of age, with Non-Alcoholic Steatohepatitis (NASH) confirmed by liver biopsy performed in a period of 6 months before entering the study, with overweight or obesity and who are pre diabetic or type II diabetic.

### Intervention

Drug: Aramchol

Subjects will be administered Aramchol as follows:
a. One tablet of Aramchol 400 mg and one tablet of matching placebo for Aramchol.
b. One tablet of Aramchol 400 mg and one tablet of Aramchol 200 mg.
c. Two tablet of Aramchol matching placebo. The tablets should be taken orally in the morning within 30 min after breakfast with a glass of water (230 ml).

Subjects are allowed to omit study drugs up to 3 consecutive days during the study.

Other Name: Placebo

### Study Arms

Experimental: Aramchol 600 mg
a. One tablet of Aramchol 400 mg and one tablet of Aramchol 200 mg.
b. Intervention: Drug: Aramchol

Experimental: Aramchol 400 mg
a. One tablet of Aramchol 400 mg and one tablet of matching placebo for Aramchol.
b. Intervention: Drug: Aramchol

Placebo comparator: Placebo
a. Two tablet of Aramchol matching placebo.
b. Intervention: Drug: Aramchol

### Estimated Enrollment 240

### Inclusion Criteria

Male or female age 18 to 75 years.

BMI between 25 kg/m2 to 40 kg/m2 or waist circumference between 88 cm to 200 cm for women, and between 102 cm to 200 cm for men. If there is deviation above the upper limit, please consult the MRI center, to ensure that the machine is suitable for the patient.

Known type II Diabetes Mellitus or pre-Diabetes according to American Diabetes Association. One of the following 3 criteria is needed for pre-Diabetes: Fasting Plasma Glucose>100 mg/dl (5.5 mmol/l) or 2hPG following 75 g OGTT>140 (7.8 mmol/l) mg/dl of HbA1c>5.7%. HbA1c can be repeated at Investigator's discretion.

Histologically proven Steatohepatitis on a diagnostic liver biopsy performed either during screening or within 6 months before screening visit, confirmed by central laboratory reading of the slides. (Steatosis≥1+inflammation≥+ballooning≥1). Total activity HAS score of 4 or more.

Liver fat concentration in the liver of 5.5% or more as measured by NMRS.

Biopsies with an activity NAS score of 4 or more.

Normal synthetic liver function (serum albumin>3.2 g/dl, INR 0.8-1.2, conjugated bilirubin<35 μmol/L).

Understanding the nature of the study and signature of the written informed consent.

Negative pregnancy test at study entry for females of child tearing potential.

Females of child bearing potential practicing reliable contraception throughout the study period (including oral contraceptives) as well as negative pregnancy test at study entry.

Hypertensive patients must be well controlled by stable dose of anti-hypertensive medication for at least 2 months prior to screening.

Patients previously treated with vitamin E (>400 IU/day), Polyunsaturated fatty acid (>2 g/day) or Ursodeoxycholic acid or fish oil can be included it stopped or at least maintained on stable dose at least 3 months prior to diagnostic liver biopsy (and are not started during the trial). These treatments-dosages are allowed if they were stable for at least 12 months prior to biopsy and can remain stable throughout the study. (Dosages less than the amounts stated above are allowed without washout- or stable-period restrictions).

For patients with type II Diabetes, glycaemia must be controlled (Glycosylated Hemoglobin A1c≤9%) while any HbA1c change should not exceed 1.5% during 6 months prior to enrollment). Treatments with anti-diabetic medications (except for those mentioned in Exclusion 16) are permitted if glycaemia is self-monitored by the patient. HbA1c can be repeated at Investigator's discretion.

### Exclusion Criteria

Exclusion Criteria:

Patients with other active (acute or chronic) liver disease other than NASH (e.g. viral hepatitis, unless eradicated at least 3 years prior to screening; genetic hemochromatosis; Wilson disease; alpha lantitripsin deficiency; alcohol liver disease; drug-induced liver disease) at the time of randomization.

Patients with clinically or histologically documented liver cirrhosis

Known alcohol and/or any other drug abuse or dependence in the last five years.

Known history or presence of clinically significant cardiovascular, gastrointestinal, metabolic other than Diabetes Mellitus, neurologic, pulmonary, endocrine, psychiatric, neoplastic disorder or nephrotic syndrome, that in the opinion of the Investigator warrant exclusion from the study.

Patients with familial (i.e., genetic) hypertriglyceridemia and familial (i.e., genetic) hypercholesterolemia.

History or presence of any disease or condition known to interfere with the absorption distribution, metabolism or excretion of drugs including bile salt metabolites (e.g. inflammatory bowel disease (IBD), previous intestinal (ileal or colonic) operation, chronic pancreatitis, celiac disease or previous vagotomy. Ongoing Chronic constipation.

Patients with heart or brain pacemaker (i.e., implantable neurological devices).

Surgery during the last three month before screening which involved stent implantation of metal devices (e.g. knee, hip etc.)

Weight less of more than 5% within months prior to randomization.

History of bariatric surgery within 5 years of liver biopsy.

Uncontrolled arterial hypertension.

Women who are pregnant and breast feeding.

Diabetes Mellitus other than type II (type I, endocrinopathy, genetic syndromes etc.).

Patients with HIV infection.

Daily alcohol intake >20 g/day for women and >30 g/day for men (on average per day) as per medical history.

Treatment with other anti-diabetic medications: GLP-1 receptor agonists and Thiazolidinediones (TZDs), unless started at least 12 months prior to biopsy and on stable dose for 6 months. In case of GLP-1 receptor agonists stopped, it should be at least 6 months before biopsy as per medical history.

SGLT-2 Inhibitors, Metformin, fibrates, statins, insulin, DP-4 inhibitors and sulfonylurea unless prescribed dose has been stable for the last 6 months prior to the biopsy.

Treatment with Valproic acid, Tamoxifen, Methotrexate, Amiodarone or chronic treatment with anti-cholinergic agents, corticosteroids, high dose estrogen and tetracycline within 12 months prior to the screening visit.

Chronic treatment with antibiotics (e.g. Rifaximin).

Homeopathic and/or alternative treatments. Any treatment should be stopped during the screening period at least 48 hours before randomization.

Uncontrolled hypothyroidism defined as Thyroid Stimulating hormone >2× the upper limit of normal (ULN). Thyroid dysfunction controlled for at least 6 months prior to screening is permitted.

Patients with renal dysfunction eGFR<40.

Unexplained serum creatine phosphokinase (CPK) >3× the upper limit of normal (UNL). Patients with a reason for CPK elevation may have the measurement repeated prior to randomization; a CPK retest >3× ULN leads to exclusion.

Patients with condition(s) that makes them unsuitable to perform the NMRS (as determined by the PI or the MRI facility).

Hypersensitivity to Aramchol or to any of the excipients in the tablets.

Hypersensitivity to cholic acid or bile acid sequestrants.

### DETAILED DESCRIPTION

This is a multicenter, Phase IIb, randomized, double blind, placebo-controlled study designed to evaluate the efficacy and safety of two Aramchol doses in subjects that are 18 to 75 years of age, with Non-Alcoholic Steatohepatitis (NASH) confirmed by liver biopsy performed in a period of 6 months before entering the study, with overweight or obesity and who are pre diabetic or type II diabetic.

Eligible subjects will be enrolled into three treatments arms: Aramchol 400 and 600 mg tablets and placebo tablets in ratio 2:2:1.

The subjects will be evaluated at study sites for 11 scheduled visits: at screening (visit 1 (weeks −4-0)), baseline (visit 2 (day 0)), visit 3 (week 2), visit 4 week 4), visit 5 (week 8), visit 6 (week 12), visit 7 (week 24), visit 8 (week 32), visit 9 (week 40) and visit 10 (week 52—(End of Treatment/early termination visit)). After completion of the study treatment period, the subjects will be followed for an additional period of 13 weeks without study medication (until visit 11 (week 65)).

During the screening period, the severity of the disease will be evaluated with blood tests, liver biopsy and NMRS.

During the study the following assessments will be performed:
a. vital signs will be measured at each study visit.
b. a physical examination will be performed at the screening visit, 24 weeks. End of Treatment/early termination and week 65 visit.

The following blood tests will be performed: complete blood count (CBC), serum chemistry (including electrolytes, liver enzymes, direct and total bilirubin, glucose, lipid profile which include triglyceride, cholesterol, HDL, LDL and VLDL, CPK, creatinine, urea, albumin, alkaline phosphatase), ESR and urinalysis during the screening visit, baseline, week 2, 4, 8, 24, 40, 52 and 65 (end of follow up) visits. Serology (HBV, HCV and HIV) will be performed during the screening visit. Coagulation (fibrinogen, PT/INR, aPTT) will be measured in screening and baseline, week 24, End of Treatment/early termination and week 65 visits. Insulin (HOMA) will be measured in the screening, week 24 and End of Treatment/early termination visits. HbA1C will be measured in the screening, week 8, 24, 40 and End of Treatment/early termination visits. C reactive protein, Leptin, Adiponectin, CK-18 (M30 and M65), Ferritin, PAI-1, IL-6, TNP-alpha, FGF-19, C4 (7-alpha-hydroxy-4-cholesten-3-one), pool serum Bile Acids, B-hydroxybutyrate and Free Fatty Acids will be measured in baseline visit and end of treatment period. The blood samples taken at these visits, will be tested for possible biomarkers, including, but not limited to, Fetuine A and GDF15. TSK, T3 and 4 will be measured during the screening visit. beta-hCG in women of childbearing potential will be performed during the screening visit. A serum sample will be collected and kept frozen until study end in case special investigation needs to be performed. This sample will be collected during the screening and visit 10/Early Termination.

Body weight and waist circumference will be measured in screening, baseline, week 24, end of treatment and week 65 visits. Height will be measured during the screening visit.

ECG will be performed during the screening visit, visit 7 (week 24) and end of treatment visits.

All subjects will undergo two NMRS scans, at screening and end of treatment visits.

FibroMax test will be performed only if the investigator thinks it is necessary.

Liver biopsy will be conducted during the screening and end of treatment visit. The biopsy in the screening visit will be performed only if it was not done within the 6 months prior to this visit.

Metabolomics blood test will be performed at the screening, visit 7 and the End-of-Treatment/Early Termination visits. From some consenting patients (about 15) a sample from the liver biopsy will be taken for analysis.

Endothelial Function will be conducted in selected sites. The test will be conducted during the baseline visit before the study treatment will be given and End of Treatment/early termination visit.

Blood sample for Aramchol trough level will be collected (pre-dose) from patients in Israel at baseline (visit 2) week 4 (visit 4), week 12 (visit 6), week 24 (visit 7), week 40 (visit 9), end of treatment (visit 10) and follow up (visit 11). At selected sites in Mexico, USA and Hong Kong one blood sample will be collected (pre-dose) on visit 4 (up to 10 subjects per country) to test for trough Aramchol blood level differences between populations (e.g., African American, Asian, Hispanic).

Blood sample for gene analysis will be taken from all consenting patients during the baseline visit, will be kept frozen and analyzed only at the study end.

Life style questionnaire will be completed in all visits.

Adverse events will be monitored throughout the study.

Concomitant Medications will be monitored throughout the study.

Telephone contacts will be performed on week 16, 20, 28, 36, 44 and 49. An interim safety analysis will be conducted as soon as 120 subjects will completed the follow up period of 24 weeks under study treatment. An Independent DSMB will analyze the safety data and recommend a continued course of action. All patients will continue to be treated under the study protocol until conclusion of the analysis will be known.

Safety assessment will include frequency and severity of treatment-emergent AEs, clinically significant laboratory abnormalities, ECG changes and physical examination findings.

### Results

### Primary and Secondary Outcome Measures (400 Arm)

Treatment with 400 mg of Aramchol significantly reduces liver triglycerides ratio as measured by Magnetic Resonance Spectroscopy (MRS).

Treatment with 400 mg of Aramchol reduces liver triglycerides ratio as measured by Magnetic Resonance Spectroscopy (MRS) by 10-40%.

Treatment with 400 mg of Aramchol reduces liver triglycerides ratio as measured by Magnetic Resonance Spectroscopy (MRS) by 15%-35%.

Treatment with 400 mg of Aramchol reduces liver triglycerides ratio as measured by Magnetic Resonance Spectroscopy (MRS) by 20%-30%.

Treatment with 400 mg of Aramchol results in a significantly higher proportion of subjects having fibrosis improvement (i.e. decrease > or = to 1 point) without a worsening of NASH, compared to subjects treated with a placebo.

Treatment with 400 mg of Aramchol results in a significantly higher proportion of subjects having fibrosis Improvement (i.e. decrease > or = to 1 point) without a worsening of NASH, compared to subjects treated with a placebo. The improvement ratio is at least 2 when compared to subjects created with a placebo.

Treatment with 400 mg of Aramchol results in a significantly higher proportion of subjects with NAS Score improvement (i.e. improvement of at least 2 points) without worsening of fibrosis score, compared to subjects treated with a placebo.

Treatment with 400 mg of Aramchol results in a significantly higher proportion of subjects with SAF Activity score improvement (i.e. improvement of at least 2 points) without worsening of fibrosis score, compared to subjects treated with a placebo.

Treatment with 400 mg of Aramchol results in a significantly higher proportion of subjects with NASH resolution (ballooning of 0, inflammation of 0 or 1) without worsening of fibrosis, compared, to subjects treated with a placebo.

### Exploratory Outcome Measures (400 mg Arm)

Treatment with 400 mg of Aramchol inhibits worsening of the subject's fibrosis score significantly more than what would be expected based on Aramchol's effect on the subject's liver triglycerides.

Treatment with 400 mg of Aramchol to subjects afflicted with hepatic fibrosis improves the subject's fibrosis score significantly more than what would be expected based on Aramchol's effect on the subject's liver triglycerides.

Treatment with 400 m of Aramchol to subjects afflicted with stage 1a hepatic fibrosis improves the subject's fibrosis score significantly more than the effect that would be expected based on Aramchol'3 effect on the subject's liver triglycerides.

Treatment with 400 mg of Aramchol to subjects afflicted with stage 1b hepatic fibrosis improves the subject's fibrosis score significantly more than the effect that would be expected based on Aramchol's effect on the subject's liver triglycerides.

Treatment with 400 mg of Aramchol to subjects afflicted with stage 1c hepatic fibrosis improves the subject's fibrosis score significantly more than the effect that would be expected based on Aramchol's effect on the subject's liver triglycerides.

Treatment with 400 mg of Aramchol to subjects afflicted with stage 2 hepatic fibrosis improves the subject's fibrosis score significantly more than the effect that would be expected based on Aramchol's effect on the subject's liver triglycerides.

Treatment with 400 mg of Aramchol to subjects afflicted with stage 3 hepatic fibrosis improves the subject's fibrosis score significantly more than the effect that would be expected based on Aramchol effect on the subject's liver triglycerides.

Treatment with 400 mg of Aramchol to subjects afflicted with stage 4 hepatic fibrosis improves the subject's fibrosis score significantly more than the effect that would be expected based on Aramchol's effect on the subject's liver triglycerides.

Treatment with 400 mg of Aramchol to subjects afflicted with hepatic fibrosis improves the subject's SAF score more than what would be expected based on Aramchol's effect on the subject's liver triglycerides.

Treatment with 400 mg of Aramchol to subjects afflicted with hepatic fibrosis results in a significantly higher proportion of subjects without worsening of fibrosis score, compared to subjects afflicted with hepatic fibrosis treated with a placebo.

Treatment with 400 mg of Aramchol to subjects afflicted with hepatic fibrosis results in a significantly higher proportion of subjects having fibrosis improvement (i.e. decrease > or = to 1 point), compared to subjects afflicted with hepatic fibrosis treated with a placebo.

Treatment with 400 mg of Aramchol to subjects afflicted with hepatic fibrosis results in a significantly higher proportion of subjects having fibrosis improvement (i.e. decrease > or − to 1 point) without a worsening of NASH, compared to subjects afflicted with hepatic fibrosis treated with a placebo.

Treatment with 400 mg of Aramchol to subjects afflicted with hepatic fibrosis results in a significantly higher proportion of subjects having fibrosis improvement (i.e. decrease > or − to 1 point) without a worsening of NASH, compared to subjects treated with a placebo. The improvement ratio is at least 2 when compared to subjects afflicted with hepatic fibrosis treated with a placebo.

Treatment with 400 mg of Aramchol results in a significantly higher proportion of subjects with NAS Score improvement (i.e. improvement of at least 2 points) compared to subjects afflicted with hepatic fibrosis treated with a placebo.

Treatment with 400 mg of Aramchol results in a significantly higher proportion of subjects with NAS score improvement (i.e. improvement of at least 2 points) without worsening, of fibrosis score, compared to subjects afflicted with hepatic fibrosis treated with a placebo.

Treatment with 400 mg of Aramchol to subjects afflicted with hepatic fibrosis results in a significantly higher proportion of subjects with SAF Activity score improvement (i.e. improvement of at least 2 points) compared to subjects afflicted with hepatic fibrosis treated with a placebo.

Treatment with 400 mg of Aramchol to subjects afflicted with hepatic fibrosis results in a significantly higher proportion of subjects with SAF Activity score improvement (i.e. improvement of at least 2 points) without worsening of fibrosis score, compared to subjects afflicted with hepatic fibrosis treated with a placebo.

Treatment with 400 mg of Aramchol to subjects afflicted with hepatic fibrosis results in a significantly higher proportion of subjects with NASH resolution (ballooning of 0, inflammation of 0 or 1) compared to subjects afflicted with hepatic fibrosis treated with a placebo.

Treatment with 400 mg of Aramchol to subjects afflicted with hepatic fibrosis results in a significantly higher proportion of subjects with NASH resolution (ballooning of 0, inflammation of 0 or 1) without worsening of fibrosis, compared to subjects afflicted with hepatic fibrosis treated with a placebo.

Treatment with 400 mg of Aramchol to subjects not afflicted with hepatic fibrosis results in a significantly higher proportion of subjects without worsening of fibrosis score, compared to subjects not afflicted with hepatic fibrosis treated with a placebo.

Treatment with 400 mg of Aramchol results in a significantly higher proportion of subjects with NAS Score improvement (i.e. improvement of at least 2 points) compared to subjects not afflicted with hepatic fibrosis treated with a placebo.

Treatment with 400 mg of Aramchol results in a significantly higher proportion of subjects with NAS Score improvement (i.e. improvement of at least 2 points) without worsening of fibrosis score, compared to subjects not afflicted with hepatic fibrosis created with a placebo.

Treatment with 400 mg of Aramchol to subjects net afflicted with hepatic fibrosis results in a significantly higher proportion of subjects with SAF Activity score improvement (i.e. improvement of at least 2 points) compared to subjects not afflicted with hepatic fibrosis treated with a placebo.

Treatment with 400 mg of Aramchol to subjects not afflicted with hepatic fibrosis results in a significantly higher proportion of subjects with SAF Activity score improvement (i.e. improvement of at least 2 points) without worsening of fibrosis score, compared to subjects not afflicted with hepatic fibrosis treated with a placebo.

Treatment with 400 mg of Aramchol to subjects not afflicted with hepatic fibrosis results in a significantly higher proportion of subjects with NASH resolution (ballooning of 0, inflammation of 0 or 1) compared to subjects not afflicted with hepatic fibrosis treated with a placebo.

Treatment with 400 mg of Aramchol to subjects not afflicted with hepatic fibrosis results in a significantly higher proportion of subjects with NASH resolution (ballooning of 0, inflammation of 0 or 1) without worsening of fibrosis, compared to subjects not afflicted with hepatic fibrosis treated with a placebo.

### Primary and Secondary Outcome Measures (600 mg Arm)

Treatment with 600 mg of Aramchol significantly reduces liver triglycerides ratio as measured by Magnetic Resonance Spectroscopy (MRS).

Treatment with 600 mg of Aramchol reduces liver triglycerides ratio as measured by Magnetic Resonance Spectroscopy (MRS) by 10-40%.

Treatment with 600 mg of Aramchol reduces liver triglycerides ratio as measured by Magnetic Resonance Spectroscopy (MRS) by 15%-35%.

Treatment with 600 mg of Aramchol reduces liver triglycerides ratio as treasured by Magnetic Resonance Spectroscopy (MRS) by 20%-30%.

Treatment with 600 mg of Aramchol results in a significantly higher proportion of subjects having fibrosis improvement (i.e. decrease > or = to 1 point) without a worsening of NASH, compared to subjects treated with a placebo.

Treatment with 600 mg of Aramchol results in a significantly higher proportion of subjects having fibrosis improvement (i.e. decrease > or = to 1 point) without a worsening of NASH, compared to subjects treated with a placebo. The improvement ratio is at least 2 when compared to subjects treated with a placebo.

Treatment with 600 mg of Aramchol results in a significantly higher proportion of subjects with NAS Score improvement (i.e. improvement of at least 2 points) without worsening of fibrosis score, compared to subjects treated with a placebo.

Treatment with 600 mg of Aramchol results in a significantly higher proportion of subjects with SAF Activity score Improvement (i.e. improvement of at least 2 points) without worsening of fibrosis score, compared to subjects created with a placebo.

Treatment with 600 mg of Aramchol results in a significantly higher proportion of subjects with NASH resolution (ballooning of 0, inflammation of 0 or 1) without worsening of fibrosis, compared to subjects treated with a placebo.

### Exploratory Outcome Measures (600 mg Arm)

Treatment with 600 mg of Aramchol inhibits worsening of the subject's fibrosis score significantly more than what would be expected based on Aramchol's effect on the subject's liver triglycerides.

Treatment with 600 mg of Aramchol to subjects afflicted with hepatic fibrosis improves the subject's fibrosis score significantly mere than what would be expected based on Aramchol's effect on the subject's liver triglycerides.

Treatment with 600 mg of Aramchol to subjects afflicted with stage 1a hepatic fibrosis improves the subject's fibrosis score significantly more than the effect that would be expected based on Aramchol's effect on the subject's liver triglycerides.

Treatment with 600 mg of Aramchol to subjects afflicted with stage 1b hepatic fibrosis improves the subject's fibrosis score significantly more than the effect that would be expected based on Aramchol's effect on the subject's liver triglycerides.

Treatment with 600 mg of Aramchol to subjects afflicted with stage 1c hepatic fibrosis improves the subject's fibrosis score significantly more than the effect, that would be expected based on Aramchol's effect on the subject's liver triglycerides.

Treatment with 600 mg of Aramchol to subjects afflicted with stage 2 hepatic fibrosis improves the subject's fibrosis score significantly wore than the effect that would be expected based on Aramchol's effect on the subject's liver triglycerides.

Treatment with 600 mg of Aramchol to subjects afflicted with stage 3 hepatic fibrosis improves the subject's fibrosis score significantly more than the effect that would be expected based on Aramchol's effect on the subject's liver triglycerides.

Treatment with 600 mg of Aramchol to subjects afflicted with stage 4 hepatic fibrosis improves the subject's fibrosis score significantly more than the effect that would be expected based on Aramchol's effect on the subject's liver triglycerides.

Treatment with 600 mg of Aramchol to subjects afflicted with hepatic fibrosis improves the subject's SAF score mere than what would be expected based on Aramchol's effect on the subject's liver triglycerides.

Treatment with 600 mg of Aramchol to subjects afflicted with hepatic fibrosis results in a significantly higher proportion of subjects without worsening of fibrosis score, compared to subjects afflicted with hepatic fibrosis treated with a placebo.

Treatment with 600 mg of Aramchol to subjects afflicted with hepatic fibrosis results in a significantly higher proportion of subjects having fibrosis improvement (i.e. decrease > or = to 1 point), compared to subjects afflicted with hepatic fibrosis treated with a placebo.

Treatment with 600 mg of Aramchol to subjects afflicted with hepatic fibrosis results in a significantly higher proportion of subjects having fibrosis improvement (i.e. decrease > or = to 1 point) without a worsening of NASH, compared to subjects afflicted with hepatic fibrosis treated with a placebo.

Treatment with 600 mg of Aramchol to subjects afflicted with hepatic fibrosis results in a significantly higher proportion of subjects having fibrosis improvement (i.e. decrease > or = to 1 point) without a worsening of NASH, compared to subjects treated with a placebo. The improvement ratio is at least 2 when compared to subjects afflicted with hepatic fibrosis treated with a placebo.

Treatment with 600 mg of Aramchol results in a significantly higher proportion of subjects with NAS Score Improvement (i.e. improvement of at least 2 points) compared to subjects afflicted with hepatic fibrosis treated with a placebo.

Treatment with 600 mg of Aramchol results in a significantly higher proportion of subjects with NAS Score improvement (i.e. improvement of at least 2 points) without worsening of fibrosis score, compared to subjects afflicted with hepatic fibrosis treated with a placebo.

Treatment with 600 mg of Aramchol to subjects afflicted with hepatic fibrosis results in a significantly higher proportion of subjects with SAF Activity score improvement (i.e. improvement of at least 2 points) compared to subjects afflicted with hepatic fibrosis treated with a placebo.

Treatment with 600 mg of Aramchol to subjects afflicted with hepatic fibrosis results in a significantly higher proportion of subjects with SAF Activity score improvement (i.e. improvement of at least 2 points) without worsening of fibrosis score, compared to subjects afflicted with hepatic fibrosis treated with a placebo.

Treatment with 600 mg of Aramchol to subjects afflicted with hepatic fibrosis results in a significantly higher proportion of subjects with NASH resolution (ballooning of 0, inflammation of 0 or 1) compared to subjects afflicted with hepatic fibrosis treated with a placebo.

Treatment with 600 mg of Aramchol to subjects afflicted with hepatic fibrosis results in a significantly higher proportion of subjects with NASH resolution (ballooning of 0, inflammation off 0 or 1) without worsening of fibrosis, compared to sublets afflicted with hepatic fibrosis treated with a placebo.

Treatment with 600 mg of Aramchol to subjects not afflicted with hepatic fibrosis results in a significantly higher proportion of subjects without worsening of fibrosis score, compared to subjects rot afflicted with hepatic fibrosis treated with a placebo.

Treatment with 600 mg of Aramchol results in a significantly higher proportion of subjects with NAS Score improvement (i.e. improvement of at least 2 points) compared to subjects not afflicted with hepatic fibrosis treated with a placebo.

Treatment with 600 mg of Aramchol results in a significantly higher proportion of subjects with NAS Score improvement (i.e. improvement of at least 2 points) without worsening of fibrosis score, compared to subjects net afflicted with hepatic fibrosis treated with a placebo.

Treatment with 600 mg of Aramchol to subjects not afflicted with hepatic fibrosis results in a significantly higher proportion of subjects with SAF Activity score improvement (i.e. improvement of at least 2 points) compared to subjects not afflicted with hepatic fibrosis treated with a placebo.

Treatment with 600 mg of Aramchol to subjects not afflicted with hepatic fibrosis results in a significantly higher proportion of subjects with SAF Activity score improvement (i.e. improvement of at least 2 points) without worsening of fibrosis score, compared to subjects net afflicted with hepatic fibrosis treated with a placebo.

Treatment with 600 mg of Aramchol to subjects not afflicted with hepatic fibrosis results in a significantly higher proportion of subjects with NASH resolution (ballooning of 0, inflammation of 0 or 1) compared to subjects not afflicted with hepatic fibrosis treated with a placebo.

Treatment with 600 mg of Aramchol to subjects not afflicted with hepatic fibrosis results in a significantly higher proportion of subjects with NASH resolution (ballooning of 0, inflammation of 0 or 1) without worsening of fibrosis, compared to subjects not afflicted with hepatic fibrosis treated with a placebo.

### DISCUSSION

Based on studies described herein, Aramchol is surprisingly found to be a potent anti-fibrotic and anti-cirrhotic agent. Aramchol is was also found to be unexpectedly superior to OCA and provides improved, effective treatment for liver fibrosis. Accordingly, Aramchol may be used to prevent acute or fatal liver failure and/or hepatic or portosystemic encephalopathy, for example toxin-induced liver failure and/or hepatic encephalopathy.

Furthermore, Aramchol is also surprisingly found to be effective in reversing established fibrosis. Aramchol treatment improves liver histology as determined by a reduction of lipid accumulation (Sudan red staining), fibrosis (Sirius red and SMA staining) and inflammation (F4/80 and CD64 staining). Indeed. Aramchol has an effect on fibrosis in addition to main pathologies of NASH, namely steatosis and inflammation.

Results presented herein show that Aramchol down-regulates collagen production from human stellate cells, the effects of Aramchol are mediated through down regulation of SCD 1 and up regulation of glutathione production, and the effect of Aramchol on fibrosis is mediated via down regulation of steatosis and inflammation as well as directly via down regulation of collagen production from stellate cells. Taken together, information herein supports the effects of Aramchol in human patients as set forth in the claims.

Results analogous to those of Example 4 for 400 mg or 600 mg doses are expected for higher doses of Aramchol that are recited herein.

1. A method for inhibiting the development of hepatic fibrosis in a human subject afflicted with Non-Alcoholic Fatty Liver Disease and having a fibrosis score of zero comprising orally administering to the subject greater than 300 mg per day of 3β-arachidylamido-7α, 12α-dihydroxy-5β-cholan-24-oic acid (Aramchol), or a pharmaceutically acceptable salt thereof, thereby inhibiting the development of hepatic fibrosis in said subject.

2. The method of claim 1 wherein the human subject is afflicted with Non-Alcoholic Steatohepatitis (NASH).

3. The method of claims 2 wherein the human subject has a NAFLD Activity (NAS) Score of at least 4, at least 5, at least 6, or at least 7; or the human subject has a ballooning score of at least 1, an inflammation score of at least 1, and a steatosis score of at least 1; or the human subject is afflicted with Diabetes Mellitus type II or pre-diabetes, or any combination thereof.

4. (canceled)

5. The method of claim 1 wherein the human subject is not afflicted with Non-Alcoholic Steatohepatitis (NASH).

6. (canceled)

7. The method of claim 1 wherein between 350 mg and 1200 mg of Aramchol is administered to the subject per day.

8. The method of claim 1 wherein 400 mg or 600 mg of Aramchol is administered to the subject per day.

9. (canceled)

10. (canceled)

11. The method of claim 1 wherein the Aramchol is administered with water, or at the same time as, or within 30 minutes of a meal;
preferably wherein the meal is breakfast, lunch, or dinner;
more preferably wherein the meal is a high fat meal or
a high calorie meal.

12. The method of claim 1 wherein the Aramchol is administered over the course of at least 52 weeks, at least 72 weeks, at least 96 weeks, at least 2 years, at least 3 years, or at least 4 years.

13. The method of claim 1 wherein the administration of Aramchol prevents worsening the subject's NAFLD Activity (NAS) score, or
prevents worsening the subject's Steatosis, Activity and Fibrosis (SAF) Activity score, or
improves the subject's Steatosis, Activity and Fibrosis (SAF) Activity score;
reduces liver fat in said subject,
inhibits progression of Non-Alcoholic Fatty Liver Disease (NAFLD) and/or Non-Alcoholic Steatohepatitis (NASH), or any combination thereof.

14. (canceled)

15. (canceled)

16. The method of claim 13 wherein the subject's NAS score is at least 4 at the commencement of administration of Aramchol and the improvement of the subject's NAS score is an improvement of at least 2 points, contributes by more than one parameter;
b) the subject's SAF Activity score is at least 4 at the commencement of administration of Aramchol and improvement of the subject's SAF Activity score is an improvement of at least 2 points.

17. The method of claim 1 wherein inhibiting the development of hepatic fibrosis comprises reducing progression of hepatic fibrosis relative to a patient not treated with Aramchol.

18. (canceled)

19. (canceled)

20. The method of claim 1 wherein the human subject is afflicted with Non-Alcoholic Steatohepatitis (NASH) and the administration of Aramchol inhibits progression of NASH.

21. The method of claim 20 wherein inhibiting progression of NASH comprises prevention of progression, or reduced progression relative to a patient not treated with Aramchol.

22. The method of claim 5 wherein the human subject is not afflicted with Non-Alcoholic Steatohepatitis (NASH) at commencement of administration and the administration of Aramchol inhibits progression from Non-Alcoholic Fatty Liver Disease (NAFLD) to NASH.

23. (canceled)

24. The method of claim 1 wherein the human subject is afflicted with Non-Alcoholic Steatohepatitis (NASH) and the administration of Aramchol resolves NASH in the subject.

25. The method of claim 24 wherein NASH resolution comprises the human subject having a ballooning score of 0 and an inflammation score of 0 or 1.

26. (canceled)

27. The method of claim 1 wherein the administration of Aramchol reduces the ratio of liver triglycerides to water in the subject relative to the ratio at the commencement of administration of Aramchol, as measured by MRS.

28. The method of claim 27 wherein there is a 10% to 40% reduction in ratio of liver triglycerides to water.

29. The method of claim 1 wherein the administration of Aramchol:
a) reduces the level of Hemoglobin A1C or HOMA-IR;
b) reduces the level of Fibrinogen, CK-18, C-reactive protein (CRP), TNFα, IL 6 and fibrosis Tests (NFS;
c) reduces the ratio of leptin to diponectin; or
d) increase in the level of diponectin; in the subject relative to the level or ratio at the commencement of administration
e) reduces the human subject's body weight relative to the human subject's body weight at the commencement of administration of Aramchol;
f) reduces the human subject's waist circumference relative to the human subject's waist circumference at the commencement of administration of Aramchol; or
g) reduces the human subject's Fatty Liver Index relative to the human subject's Fatty Liver Index at the commencement of administration of Aramchol.

30. The method of claim 1 wherein the human subject has a diet that is high fat and high calorie; and/or
is resistant to lifestyle intervention
or is resistant to diet intervention.

31. The method of claim 1, wherein the subject is nave to Aramchol treatment.

32. The method of claim 31, wherein the subject is naïve to NAFLD treatment.

33-36. (canceled)
